(19) United States
(12) Patent Application Publication
(10) Pub. No.: US 2022/0199208 A1
(43) Pub. Date: Jun. 23, 2022

(54) SYSTEM AND METHOD OF MANAGING ACCESS OF A USER'S HEALTH INFORMATION STORED OVER A HEALTH CARE NETWORK

(71) Applicant: Patientory, Inc., Atlanta, GA (US)

(72) Inventor: Chrissa Tanelia McFarlane, Atlanta, GA (US)

(21) Appl. No.: 17/607,178

(22) PCT Filed: Jun. 10, 2019
(86) PCT No.: PCT/US19/36422

§ 371 Date: Oct. 28, 2021

Related U.S. Application Data
(60) Provisional application No. 62/683,513, filed on Jun. 11, 2018.
(60) Provisional application No. 62/683,524, filed on Jun. 11, 2018.
(60) Provisional application No. 62/683,537, filed on Jun. 11, 2018.
(60) Provisional application No. 62/683,556, filed on Jun. 11, 2018.
(60) Provisional application No. 62/683,568, filed on Jun. 11, 2018.

(51) Int. Cl. G16H 10/60

(52) U.S. Cl. CPC G16H 10/60

(57) ABSTRACT

A system and a method for managing access of a user's health information stored over a health care network are disclosed. The method includes providing a Health Information Exchange (HIE) server implemented over a blockchain network. The HIE server stores users' health information. Further, a user device may be present in communication with the HIE server. An access of the user's health information may be provided to a provider based on access rights assigned for the provider, in a smart contract set by a user, and a monetary value set for the access rights may be received.

### CROSS REFERENCE TO RELATED APPLICATIONS

The present disclosure is related and claims priority under 35 U.S.C. § 1.119(e) to U.S. provisional application No. 62/683,513, entitled SYSTEM AND METHOD FOR MANAGING PAYMENTS FOR ACCESSING PATIENTS INFORMATION; 62/683,524, entitled SYSTEM AND METHOD OF CONTROLLING ACCESS OF A USERS HEALTH INFORMATION STORED OVER A HEALTH CARE NETWORK; 62/683,537, entitled SYSTEM AND METHOD FOR REGULATING A VALUE OF A CRYPTOCURRENCY USED IN A HEALTH CARE NETWORK, 62/683,556, entitled SYSTEM AND METHOD FOR FACILITATING PAYMENT REQUESTS WITHIN A HEALTH CARE NETWORK, and 62/683,568, entitled SYSTEM AND METHOD OF MANAGING ACCESS OF A USERS HEALTH INFORMATION STORED OVER A HEALTH CARE NETWORK, all filed on Jun. 11, 2018, to Chrissa Tanelia McFarlane, the contents of all of which are hereby incorporated by reference in their entirety, for all purposes.

### FIELD OF THE DISCLOSURE

The present disclosure is generally related to record access management, and more particularly related to access management of a user's health information stored over a health care server implemented over a blockchain network.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner The blocks are added to the blockchain in a linear and chronological order. Cryptocurrencies are utilized over such blockchain networks to avoid the fundamental problems associated with general currencies such as double usage.

Conventional techniques for sharing medical data of a user with interested third parties involves a lot of the user's time. Further, there does not exist a common platform where the user could easily manage and allow access of his data with interested third parties, particularly where different access rights could be assigned to each interested user. Further, a method and a system utilizing which, the user could charge for allowing access of his data, is also desired.

### SUMMARY

In a first embodiment, a computer-implemented method for managing access of a user's health information stored in a healthcare network includes configuring a health information exchange server implemented over a blockchain network to store a users' health information. The computer-implemented method also includes configuring the health information exchange server to communicate with a user device present in the healthcare network, providing, through a smart contract set by a user, an access of the user's health information to a provider, based on an access right assigned to the provider in the smart contract, and receiving a monetary value set for the access right.

In a second embodiment, a system for managing access of a user's health information stored in a healthcare network includes a memory circuit storing instructions, and one or more processors configured to execute the instructions. The instructions cause the system to configure a health information exchange server implemented over a blockchain network to store a users' health information, to configure the health information exchange server to communicate with a user device present in the healthcare network, to provide, through a smart contract set by a user, an access of the user's health information to a provider, based on an access right assigned to the provider in the smart contract, and to receive a monetary value set for the access right.

In yet other embodiments, a computer-implemented method for accessing a patient's health information in a healthcare network, includes receiving, in a record access module, a request to access data from a provider and verifying that the provider has authorization to access the data, based on a smart contract in a contracts database. The computer-implemented method also includes retrieving a contract factor from the smart contract, the contract factor based on the request to access the data, calculating a contract rate based on the contract factor, providing the contract rate to the provider for approval, providing the contract rate to the record access module when the provider approves the contract rate, and allowing the record access module to provide the provider an access to the data.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a network connection diagram 100 of a Health Information Exchange (HIE) server 102, according to various embodiments.

FIG. 2A illustrates a method for symmetric encryption of data, according to various embodiments.

FIG. 2B illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates an example of a system for storing and accessing data in a health care network implemented specifically over a blockchain network, according to various embodiments.

FIG. 6 illustrates an example of a flow chart showing functioning of a record access module, according to various embodiments.

FIG. 7 illustrates an example of a flow chart showing functioning of a dynamic contract module, according to various embodiments.

FIG. 8 illustrates an example of a flowchart showing functioning of a medical records application, according to various embodiments.

FIG. 9 illustrates an example of a flowchart showing functioning of a provider access module, according to various embodiments.

FIG.10 illustrates an example of a flowchart showing functioning of a research access module, according to various embodiments.

FIG. 11 is a block diagram that illustrates a computer system used to perform at least some of the steps and methods in accordance with various embodiments.

### DETAILED DESCRIPTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred systems and methods are now described.

Current systems and methods for storing and managing transfer of health information between multiple parties in the healthcare system are often centralized structures subject to hacking, and yet mired in strict security regulations and onerous overhead costs. This state of affairs leads to a lack of efficient and transparent information exchange, to the ultimate detriment of patients and physicians Embodiments as disclosed herein resolve the above technical problem arising in the realm of healthcare data management by implementing a blockchain infrastructure to minimize security breaches and facilitate coordination between multiple entities and organizations, thus improving the health outcomes for patients.

In some embodiments, a blockchain infrastructure as disclosed herein allows the care providers to avoid medication errors, thus reducing the need for duplicate testing. Further, blockchain technology as disclosed herein effectively tracks and time stamps activities related to health information data. Thus, some embodiments provide a robust audit trail that ensures access to all interested and authorized parties to an updated version of a medical record.

Furthermore, in some embodiments, a blockchain network as disclosed herein includes smart contracts configured with universal parameters. Accordingly, patients become the primary intermediaries for sending and receiving health information. Records stored in a blockchain network as disclosed herein are robust to tampering or error, and stored across multiple participating users (e.g., the entire blockchain network). Accordingly, recovery contingencies are unnecessary. Moreover, the transparency of a blockchain network as disclosed herein substantially reduces the number of data exchange integration points and the need for tedious reporting activities.

In some embodiments, a mobile application installed in client devices allow users to interact with the blockchain network and access features such as messaging, and access updated and accurate health information. Further, some embodiments provide tracking applications and other activity trackers to enable doctors, care providers, and other parties in the blockchain network to communicate on a single, easy to use platform. Furthermore, in some embodiments, artificial intelligence, machine learning, neural networks, and other non-linear algorithms are incorporated to store and manage data in the blockchain network.

Some embodiments provide the ability for patients and other users of the blockchain network to access tokens from an external blockchain to convert into a supported cryptocurrency for access and use of storage features.

Embodiments of the present disclosure will be described more fully hereinafter with reference to the accompanying drawings in which like numerals may represent like elements throughout the several figures, and in which various example embodiments are shown. Embodiments may, however, be embodied in many different forms and should not be construed as limited to the embodiments set forth herein. The examples set forth herein are non-limiting examples and are merely examples among other possible examples.

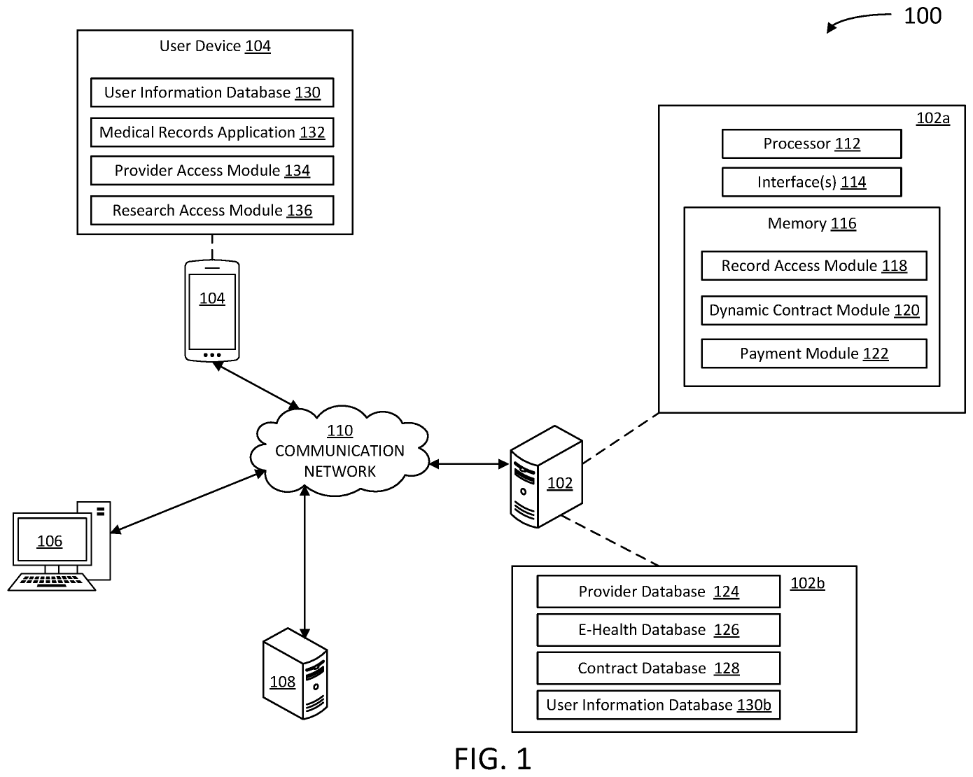

FIG. 1 illustrates an example of a network connection diagram 100 of a Health Information Exchange (HIE) server 102 for managing access of information including, for example, of a user's health information. The HIE server 102 may be connected with a user device 104, a service provider device 106, and a financial platform 108, through a communication network 110.

The communication network 110 may be a wired and/or a wireless network. The communication network 110, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE server 102 may include a group of components 102a for managing access of a user's health information. The group of components 102a may include a processor 112, interface(s) 114, a memory 116, a record access module 118, a dynamic contract module 120, and a payment module 122.

The processor 112 may execute an algorithm stored in the memory 116 for managing access of a user's health information. The processor 112 may also be configured to decode and execute any instructions received from one or more other electronic devices or server(s). The processor 112 may include one or more general-purpose processors (e.g., microprocessors) and/or one or more special purpose processors (e.g., digital signal processors or System On Chips (SOCs) Field Programmable Gate Array (FPGA) processor, Application-Specific Integrated Circuits (ASICs)). The processor 112 may be configured to execute one or more computer-readable program instructions, such as program instructions to carry out any of the functions described in this description.

The interface(s) 114 may help an operator to interact with the HIE server 102. The interface(s) 114 may either accept inputs from users or provide outputs to the users, or may perform both the actions. In one case, a user can interact with the interface(s) 114 using one or more user-interactive objects and devices. The user-interactive objects and devices may include, for example, user input buttons, switches, knobs, levers, keys, trackballs, touchpads, cameras, microphones, motion sensors, heat sensors, inertial sensors, touch sensors, or a combination of the above. Further, the interface(s) 114 may be implemented, for example, as a Command Line Interface (CLI), a Graphical User Interface (GUI), a voice interface, or a web-based user-interface.

The memory 116 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 116 may include modules implemented as a program.

In accordance with various embodiments, several users may interact with the HIE server 102, using a user device 104. Although a single user device has been illustrated, several user devices could similarly be connected to the communication network 110. Further, each of the user devices may have a device ID. For example, the device ID may be a unique identification code such as an (International Mobile Equipment Identity) IMEI code or a product serial number. It should be noted that a user may use a single user device or multiple user devices. Further, multiple users may use a single user device or multiple user devices. Further, the one or more users may receive and/or provide healthcare related products and services. The one or more users may include, for example, patients, family and friends of the patients, hospitals, physicians, nurses, specialists, pharmacies, medical laboratories, testing centers, insurance companies, or Emergency Medical Technician (EMT) services.

In accordance with various embodiments, the user device 104 may be a mobile, stationary device, a portable device, or a remote device, the visual graphical element such as, but not limited to, a barcode, text, a picture, or any other forms of graphical authentication indicia. For example, the barcode may be one-dimensional or two-dimensional. Further, the imaging device may include a hardware and/or software element. In various embodiments, the imaging device may be a hardware camera sensor that may be operably coupled to the user device 104. For example, the hardware camera sensor may be embedded in the user device 104. In another embodiment, the imaging device may be located external to the user device 104. For example, the imaging device may be connected to the user device 104 wirelessly or via a cable. It should be noted that image data of the visual graphical element may be transmitted to the user device 104 via the communication network 110.

In accordance with various embodiments, the imaging device may be controlled by applications and/or software(s) configured to scan a visual graphical code. For example, a camera may be configured to scan a QR code. Further, the applications and/or software(s) may be configured to activate the camera present in the user device 104 to scan the QR code. In various embodiments, the camera may be controlled by a processor natively embedded in the user device 104. In another case, the imaging device may include a screen capturing software (for example, screenshot) that may be configured to capture and/or scan the QR code on a screen of the user device 104.

In accordance with various embodiments, a group of databases 102b may be connected to the HIE server 102. In various embodiments, the group of databases 102b may be implemented over a blockchain network (such as a PTOYNet blockchain network or a PTOYNet Ethereum™ blockchain network), and may be present as different databases installed at different locations. The group of databases 102b may include a provider database 124, an E-health database 126, and a contract database 128. The group of databases 102b may be configured to store data belonging to different users and data for functioning of the HIE server 102. Different databases are used in present case; however, a single database may also be used for storing the data. Usage of the different databases may also allow segregated storage of different data and may thus reduce time to access data. In various embodiments, the data may be encrypted, time-dependent, piece-wise, and may be present as subsets of data belonging to each user. For an instance, the data may represent the results of one medical test in a series of multiple medical tests.

In accordance with various embodiments, the group of databases 102b may operate collectively or individually. Further, the group of databases 102b may store data as tables or charts. Further, the group of databases 102b may be configured to store data or be processed by the HIE server 102. The data may include, but is not limited to, a patient medical history, medications, prescriptions, immunizations, test results, allergies, insurance provider, or billing information. Further, the data may be time-dependent and piece-wise. Further, the data may represent a subset of data for each patient. For example, the data may represent results of a medical test in a series of multiple medical tests. Further, the data may be securely stored. In various embodiments, the data may be encrypted.

In accordance with various embodiments, information stored in the group of databases 102b may be accessed based on users' identities and/or the users' authorities. The users' identities may be verified in one or more ways such as, but not limited to, bio authentication (or biometric authentication), password or PIN information, user device registrations, a second-level authentication, or a third-level authentication. In various embodiments, the users' identities may be verified by the HIE server 102. Information provided by the users in real-time may be used, by the HIE server 102, to confirm the users' identities. For example, the users' identities may be verified using a name, a password, one or more security questions, or a combination thereof. In another example, a user may be identified using an encryption key and/or a decryption key.

In accordance with various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may need to be accessed through the first level subsystem. It should be noted that the communication between the first level subsystem and the second level subsystem may be encrypted. For example, the second level subsystem may be implemented over a PTOYNET blockchain network or a PTOYNet Ethereum™ blockchain network. In various embodiments, the PTOYNet Ethereum™ blockchain network may be used to implement smart contracts.

In accordance with various embodiments, the user device 104 includes a user information database 130, medical records application 132, provider access module 134, and a research access module 136. The medical records application 132, provider access module 134, and the research access module 136 may henceforth be used for managing access of a user's health information.

In accordance with various embodiments, a primary care physician may input data into the HIE server 102 using the user device 104. The data may be processed by the first level subsystem and the second level subsystem. This may be done successively. The data may be stored on the first level subsystem and/or the second level subsystem of the HIE server 102. This may be done successively. The data may include, but is not limited to, one or more instructions to see patient's reports. Further, the data may be stored in one or more blockchains of the second level subsystem. The patient may be able to access the data relating to the patient's care provided by the primary care physician. This may be done successively. The patient may be able to retrieve the data using the user device 104 of the patient. This may be done successively.

In accordance with various embodiments, the patient may communicate with the providers using the HIE server 102. The providers may be individuals belonging to hospitals, or doctors and insurers. It should be noted that the providers may be able to access the data of the patient from the first level subsystem and/or the second level subsystem. Further, the physician specialist may be able to communicate with the patient. It should be noted that all (or substantially all) communications between patients and providers may be stored and may be accessible on a blockchain network (such as a PTOYNet blockchain network or a PTOYNet Ethereum™ blockchain network).

Further, the HIE server 102 may include a health record network for an intermediary enabling of sharing of user's medical records with providers. For enabling sharing, the user may grant specific permissions to the providers for accessing parts of the user's medical records stored in the user information database 130b implemented over a blockchain network (such as a PTOYNet blockchain network or a PTOYNet Ethereum™ blockchain network). In various embodiments, the user may include any users constituting a value chain, such as doctors, nurses, etc. In another case, the user may be remote doctors logging into the HIE server 102 or doctors present in hospitals.

Further, the HIE server 102 may include the user information database 130b containing account information and activity of each user linked to the HIE server 102. In various embodiments, the account information and activity may include location, identifying information, and data relationships of the users with the providers. It should be noted that, the users added to the HIE server 102 should be in a direct relationship to the value of the system and hence value to the cryptocurrency.

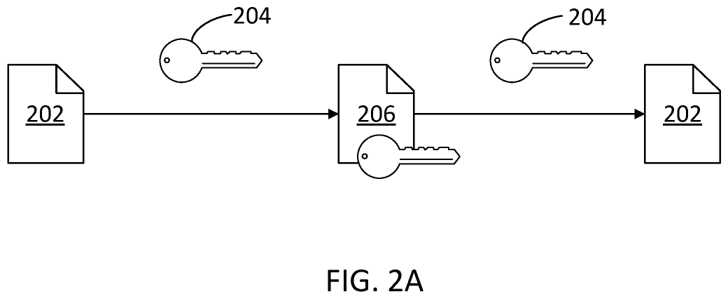

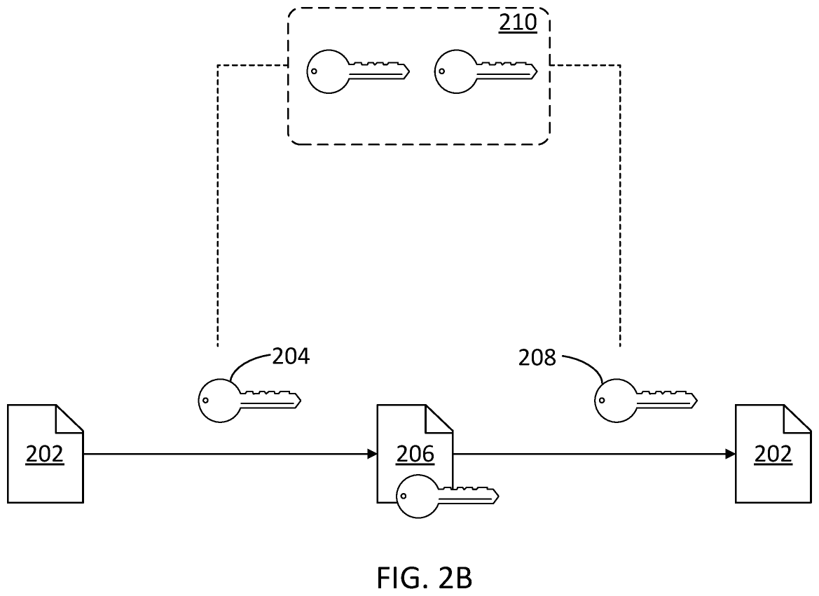

FIG. 2A illustrates a method for symmetric encryption of data, according to various embodiments. Within the method, original data 202 may be encrypted using a key 204 to obtain an encrypted data 206. Thereafter, the encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2B illustrates a method for asymmetric encryption of data, according to various embodiments. During the method, original data 202 may be encrypted using a key 204 to obtain encrypted data 206. Thereafter, the encrypted data 206 may be decrypted using another key 208 to obtain the original data 202. It should be noted that encryption and decryption of the data may be performed using different keys, i.e., a key pair 210.

In some embodiments, the steps illustrated in FIGS. 2A-B may be initiated by users who generate a new profile on the blockchain network. Private keys may be stored in decentralized and distributed hashes through the blockchain network. In some embodiments, the steps illustrated in FIGS. 2A-B may be partially performed in either one of devices 104 and 106, in HIE server 102, or financial platform 108. For example, in some embodiments, HIE server 102 may install a software development kit (SDK) or a key generator application in user device 104 or in service provider device 106 to perform at least some of the steps illustrated in FIGS. 2A-B. Likewise, keys 204, 208, and key pair 210 may be stored in a memory of either one of devices 104, 106, in HIE server 102, or in financial platform 108, or in an associated database (e.g., any one of databases 102b).

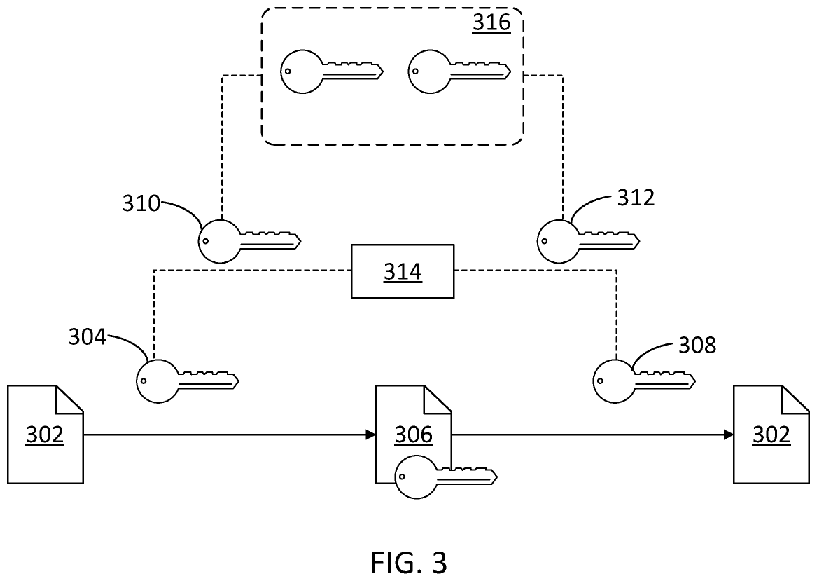

FIG. 3 illustrates a method for hybrid encryption of data, according to various embodiments. During the method, both symmetric encryption and asymmetric encryption techniques may be used in tandem. In various embodiments, the symmetric encryption technique may be used to encrypt data 302 using a symmetric key 304 for producing encrypted data 306. The encrypted data 306 may be decrypted using another symmetric key 308 for obtaining back data 302. Further, a public key 310 may be used to encrypt the symmetric key 304 and a private key 312 may be used to encrypt the symmetric key 308, stored as an encrypted key 314. The public key 310 and the private key 312 may form a key pair 316.

In some embodiments, the steps illustrated in FIG. 3 may be initiated by users who generate a new profile on the blockchain network. Private keys may be stored in decentralized and distributed hashes through the blockchain network. In some embodiments, the steps illustrated in FIG. 3 may be partially performed in either one of devices 104 and 106, in HIE server 102, or financial platform 108. For example, in some embodiments, HIE server 102 may install a software development kit (SDK) or a key generator application in user device 104 or in service provider device 106 to perform at least some of the steps illustrated in FIG. 3. Likewise, keys 204, 208, and key pair 210 may be stored in a memory of either one of devices 104, 106, in HIE server 102, or in financial platform 108, or in an associated database (e.g., any one of databases 102b).

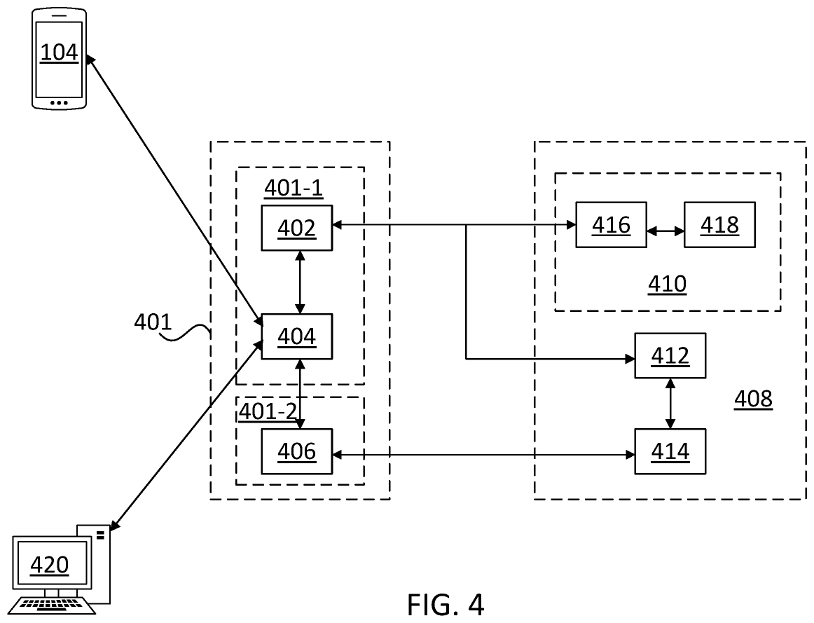

FIG. 4 illustrates a system 401 for storing and accessing data in a health care network, according to various embodiments. A first level subsystem 401-1 may include a core service component 402 and a Remote Procedure Call (RPC) component 404. A second level subsystem 401-2 may include a blockchain node 406. Blockchain node 406 may be a public node or a private node in a blockchain network having a layer over a public blockchain network, enabling the private node to perform private transactions via consensus algorithms (e.g., a Quorum blockchain node). In various embodiments, first level subsystem 401-1 may include the core service component 402, and second level subsystem 401-2 may include the RPC component 404 and the blockchain node 406. Further, the core service component 402 of first level subsystem 401-1 may be present in communication with third-party servers and databases of a hospital computing network 408. The hospital computing network 408 may include a file system module 410, an EHR synchronization service 412, and a blockchain node 414 (e.g., a Quorum blockchain node). Further, the file system module 410 may include a file system manager 416 and a file system node 418. The blockchain node 406 of second level subsystem 401-2 may communicate with the blockchain node 414 of the hospital computing network 408. Patients may access the health care network for storing data through the user device 104, and a representative of a hospital may access the health care network through another user device 420.

In accordance with various embodiments, the representative of the hospital may want to synchronize Electronic Health Record (EHR) data of a patient, e.g., by using corresponding blockchain hashes. Successively, first level subsystem 401-1 and second level subsystem 401-2 may ask the patient for permission to allow a representative of the hospital to store the EHR data of the patient, through the file system module 410. Based at least on the permission granted by the patient, a signed transaction may be created to confirm the permission of the hospital to store the EHR data. Further, the signed transaction may activate a smart contract that may add hospital identification information such as a blockchain address to a list of permitted users. In some embodiments, the signed transaction and the smart contract are stored in file system module 410.

Further, the signed transaction may be transmitted from the user device 104 to the RPC component 404 of the first level subsystem and/or the second level subsystem. The RPC component 404 may communicate the signed transaction to the blockchain node 406 of the second level subsystem. This may be done successively. The blockchain node 406 may activate one or more smart contracts. This may be done successively. Thereafter, the blockchain node 406 may revise a state of one or more blockchains.

Further, based at least on the permission granted by the patient, the EHR synchronization service may obtain a list of patients from the RPC component 404. Further, the EHR synchronization service may confirm whether the patient has granted permission. Based at least on the permission, the first level subsystem and the second level subsystem may obtain the EHR data and may calculate a hash function for the EHR data. The HIE server 102 may match the hash function of the EHR data with a hash function for the patient blockchain on the blockchain node 406 of the second level subsystem. This may be done successively. Thereafter, if the hash function of the EHR data matches with the hash function for the patient blockchain on the blockchain node 406 of the second level subsystem, the EHR data of the patient may remain unchanged.

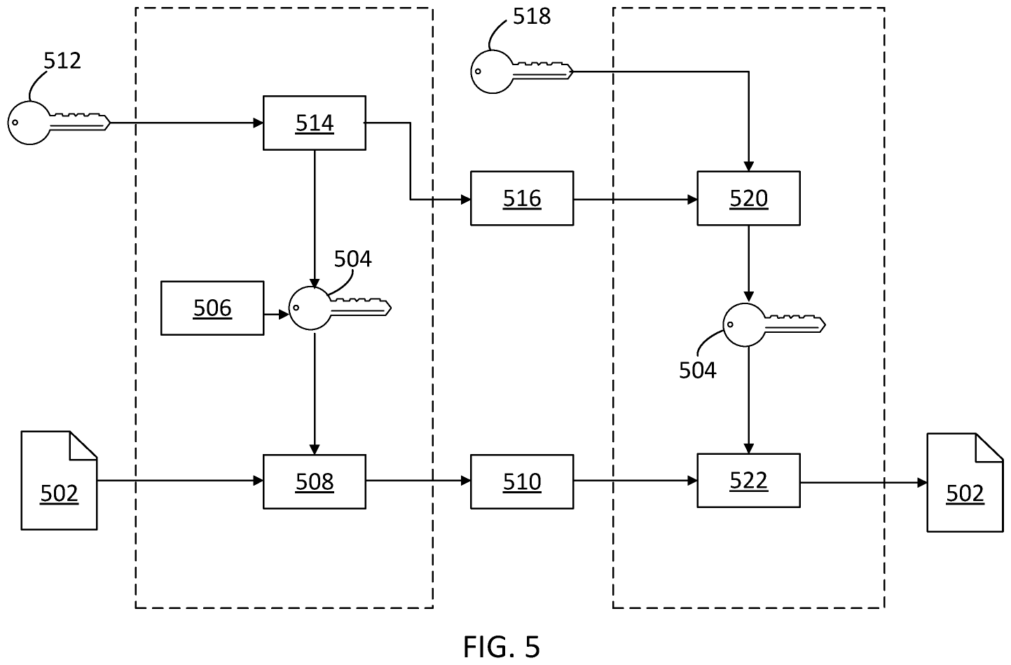

FIG. 5 illustrates a system for storing and accessing data in a health care network implemented specifically over a blockchain network as disclosed herein (cf. FIGS. 1 and 4). The HIE server 102 may execute an application for determining permission from the user for obtaining EHR data 502. In various embodiments, if the user grants the permission, the HIE server 102 may obtain the EHR data 502 for calculating a hash function for the EHR data 502. Further, the HIE server 102 may match the hash function of the EHR data 502 with a hash function for the user blockchain on the blockchain node of the second level sub-system. In various embodiments, if the two hash matches, there is no change to the user's EHR data 502. In various embodiments, if the two hash functions do not match, the HIE server 102 may generate a random string, i.e., secret key 504, through a random key generator 506. The secret key 504 may be used for Advanced Encryption Standard (AES) encryption of the EHR data 502, in an AES encryptor 508, for generating encrypted EHR data 510.

In accordance with various embodiments, the secret key 504 may then be encrypted by a Rivest-Shamir-Adleman (RSA) public key 512 of the patient, in an RSA encryptor 514, to generate an encrypted secret key 516. The HIE server 102 may further send the encrypted EHR data 510 to the core service component 402 for forwarding the data to the file system manager 416 of the hospital computing network 408 for storage. Further, the file system manager 416 may send an IPFS hash function to the core service component 402 for further sending the IPFS hash function to EHR synchronization service 412. The EHR synchronization service 412 may further update the patient smart contract with the new IPFS hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to an RPC component 404 for granting permission to the hospital representative to view the EHR data 502. Once the permission is granted, the signed transaction may be added to the blockchain node 414 and a new smart contract will be created for a blockchain corresponding to the hospital representative. After adding the signal transaction to the blockchain node, the hospital representative may be able to view the EHR data 502 of the user, on a device.

In accordance with various embodiments, in order to view the EHR data 502 on the device, the HIE server 102 may collect the encrypted EHR data 510 from the user's blockchain and may decrypt the encrypted EHR data 510 using a patient's RSA private key 518. The HIE server 102 may decrypt the encrypted secret key 516, in an RSA decryptor 520, using an RSA private key of the hospital representative. The encrypted EHR data 510 may be decrypted using the RSA public key 512 of the hospital representative, in an AES decryptor 522. This may be done successively. Further, the HIE server 102 may load the decrypted EHR data 502 to the smart contract previously created for the hospital representative.

After loading the decrypted EHR data to the smart contract, the RPC component 404 may obtain the signed transaction from the patient's user device and transmit the signed transaction to the blockchain node 406 of the second level subsystem. The blockchain node 406 may confirm ownership of the signed transaction and may execute the smart contract for the hospital representative to view the user's data. This may be done successively.

In accordance with various embodiments, the patient may decline permission for the hospital representative to have access to the EHR data 502. In such a scenario, the user through a user device may send a signed transaction revoking permission to the RPC component 404. The RPC component 404 may forward the signed transaction to the blockchain node 406 of the second level subsystem. This may be done successively. The blockchain node 406 may confirm ownership of the signed transaction and may delete the smart contract previously created to allow the hospital representative to have access to the patient's EHR data 502. This may be done successively.

In accordance with various embodiments, the HIE server 102 may include a health record network for an intermediary enabling sharing of user's medical records with providers. For enabling sharing, the user may grant specific permissions to the providers for accessing parts of the user's medical records stored in the user information database 130b. The user may also grant specific permissions to modify the user's medical records in the user information database 130b. In various embodiments, the user may include any users constituting a value chain, such as patients, doctors, nurses, etc. In various embodiments, the user may be remote doctors logging into the HIE server 102 or doctors present in hospitals.

In accordance with various embodiments, Contract Research Organization (CRO) may pay a fee to run research on the data, where each contract may be set by the system based upon the use of the patient's data and CRO need. A doctor may pay for access to the data, and later bill for that access. There are an infinite amount of ways that money may flow by medical record element, group of medical records elements, individual or group of patients, types of data to be stored, etc.

In accordance with various embodiments, the electronic health records database (E-health DB) 126 may be a centralized repository of the medical history of all the network's users. The provider database 124 may contain the identifying information, access rights, and current contract rates for all providers who access the HIE server 102. The contract database 128 may store the current variables considered for adjusting the contract rate and their relative effect on the contract rate. These are set as business rules on system initiation, and at any point if an administrator wishes to change them, but are also adjusted by Artificial Intelligence (AI) through ongoing machine learning.

The record access module 118 may be a base software module of the medical records network and grants providers access to the data of users, at rates and permission levels in the provider database 124. The dynamic contract module 120 may examine the contract terms of the provider currently accessing the system to the current contract factors in the contract database 128 to determine the provider's current contract rate. A negative response by the provider may cause an adjustment by the AI to the contract factors in the contract database 128.

The payment module 122 may complete the transaction in a cryptocurrency between the provider, the medical records network and the user, and releases the key to access the medical data only when the transaction, and all parties' identities have been authorized via the blockchain. The user device 104 may be a mobile device or computer terminal used by a system user to grant permissions to healthcare providers to their electronic health records data. The medical records application 132 may be an application through which the user interacts with the HIE server 102.

The provider access module 134 may be branch of the medical records application 132 that allows the user to select individual providers, such as their primary care physician or their local emergency room specific access rights to their medical data. The research access module 136 may be a branch of the medical records application 132 that may display opportunities to share portions of their medical data with CRO' s, pharmaceutical companies, etc., what the offered rate in cryptocurrency may be being offered in exchange for what data for what purpose. The plurality of healthcare providers may include hospitals, doctors, and insurers.

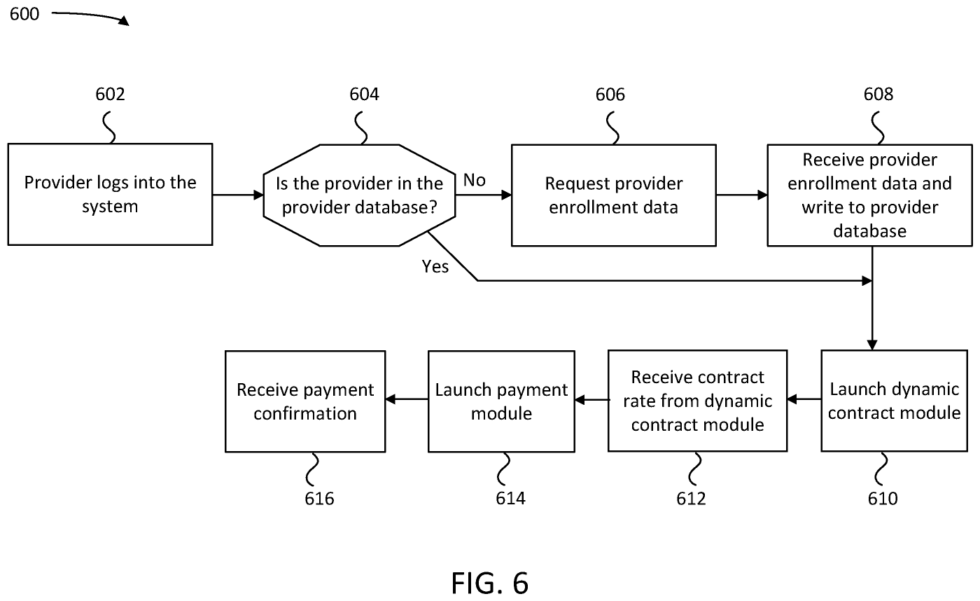

FIG. 6 illustrates a flowchart in a method 600 for operating a record access module (e.g., record access module 118), according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, a provider may login, at step 602, and availability of the provider may be checked in the provider database 124, at step 604. This may be done successively. In various embodiments, while provider details are not found, the record access module 118 may place a request for collecting provider enrolment data, such as provider name, type, and contact information, at step 606. In various embodiments, a new provider account may be created, and the provider enrollment data may be received and recorded in the new provider account created in the provider database 124, at step 608. In another case, while the provider is found to be already enrolled, at step 604, a data request may be received from the provider, at step 608. The data request may correspond to the data that the provider wishes to access.

In accordance with various embodiments, the data request received from the provider may trigger launch of the dynamic contract module 120, at step 610. This may be done successively. In various embodiments, contract rate may be received from the dynamic contract module 120, at step 612. The contract rate may be for the provider to access requested data from the dynamic contract module 120. The payment module 122 may be launched and executed, at step 614. The payment module 122 may cite related payment application, collect payment from the provider, in the form of a proprietary cryptocurrency, and authenticate the identity of the provider through the blockchain. Thereupon, a key may be released to access the agreed upon block(s) of information. A payment confirmation may be received, at step 616. This may be done successively.

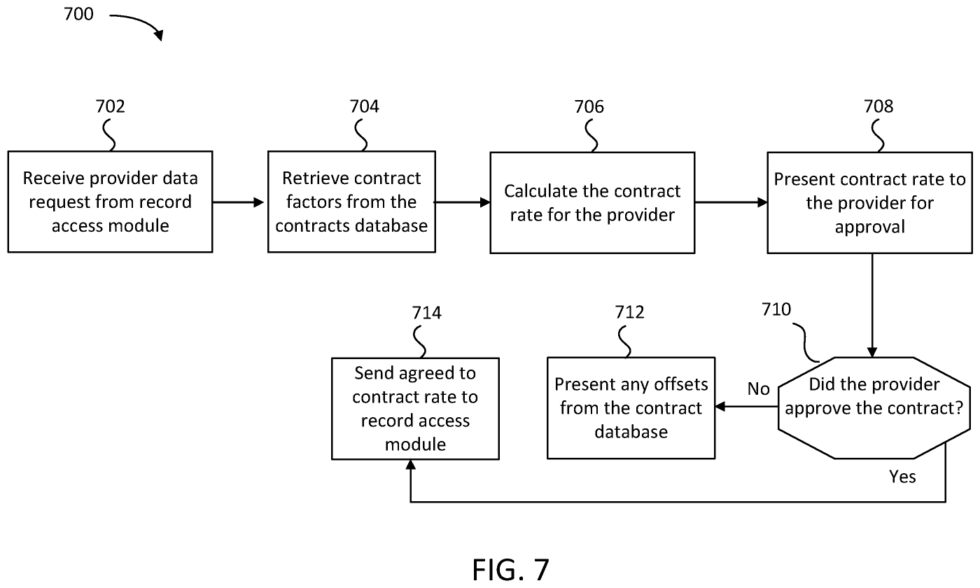

FIG. 7 illustrates a flowchart in a method 700 for operating a dynamic contract module (e. g. , dynamic contract module 120), according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

As mentioned previously, the dynamic contract module 120 may get triggered by the prompt from the record access module 118. Upon triggering, the dynamic contract module 120 receives a provider data request from the record access module 118, at step 702. The dynamic contract module 120 may retrieve contract factors from the contract database 128, at step 704. This may be done successively. The contract factors may include all factors relevant to the data being requested. In some embodiments, the contract factors include factors such as the sensitivity of requested data, value of the data, volume of requested data, as well as any offsets to the contract rate that the provider may qualify for, such as services provided to the system or user. A contract rate for the data being accessed by the provider may be calculated based on the retrieved factors, at step 706.

For example, a CRO may wish to collect data from glucometers. It may be shown that 4000 users may be willing to provide access to that data for 0.10 PTOY/month, and that number rises to 6000 users at 0.20 PTOY/month and drops to 500 users at 0.05 PTOY/month. Additionally, the CRO can get 10% of that cost paid for by the entity running the medical records network in exchange for providing 2 Terabytes of storage for the duration of the data access to be used as a storage node for the blockchain.

The contract rate may be presented to the provider for his approval, at step 708. An approval of contract by the provider may be determined, at step 710. In accordance with various embodiments, while the provider does not accept the contract, any available offsets may be displayed and the provider's selection may be polled, at step 712. In another case, while the provider accepts the contract, an agreed upon contract rate may be sent to the record access module 118, at step 714.

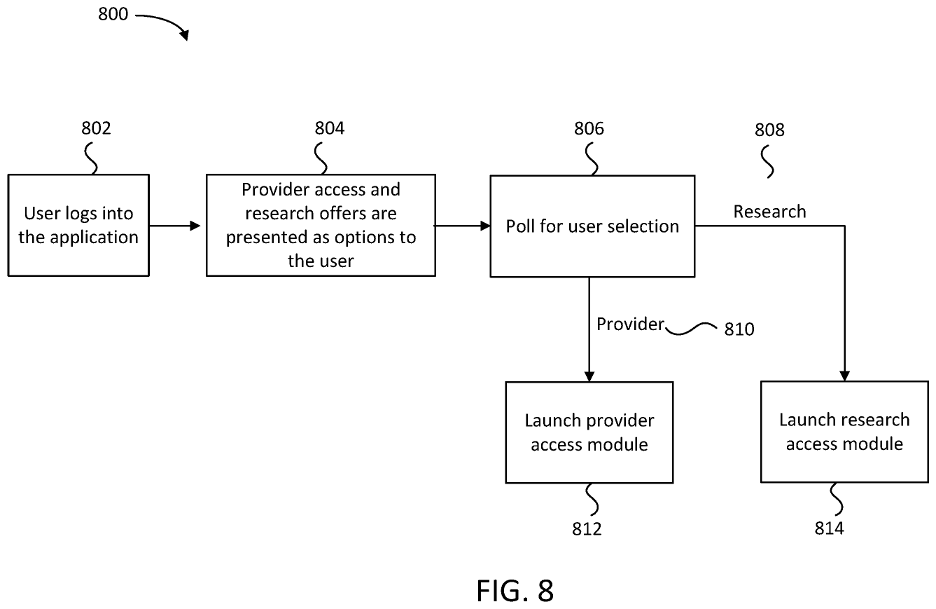

FIG. 8 illustrates a flow chart in a method 800 for operating a medical records application (e.g., medical record application 132), according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

At first, the user may login to the Medical records application 132, at step 802. Successively, two options may be presented on the user's GUI, e.g., an option to view/add providers' access and another option to view/add research offers, at step 804. Polling for the user's selection of an option may be done, at step 806. In accordance with various embodiments, the user selects provider access in step 810. Accordingly, the provider access module 134 may be executed, at step 812. In another case, while the user selects for the research offers in step 808, the research access module 136 may be launched and executed, at step 814.

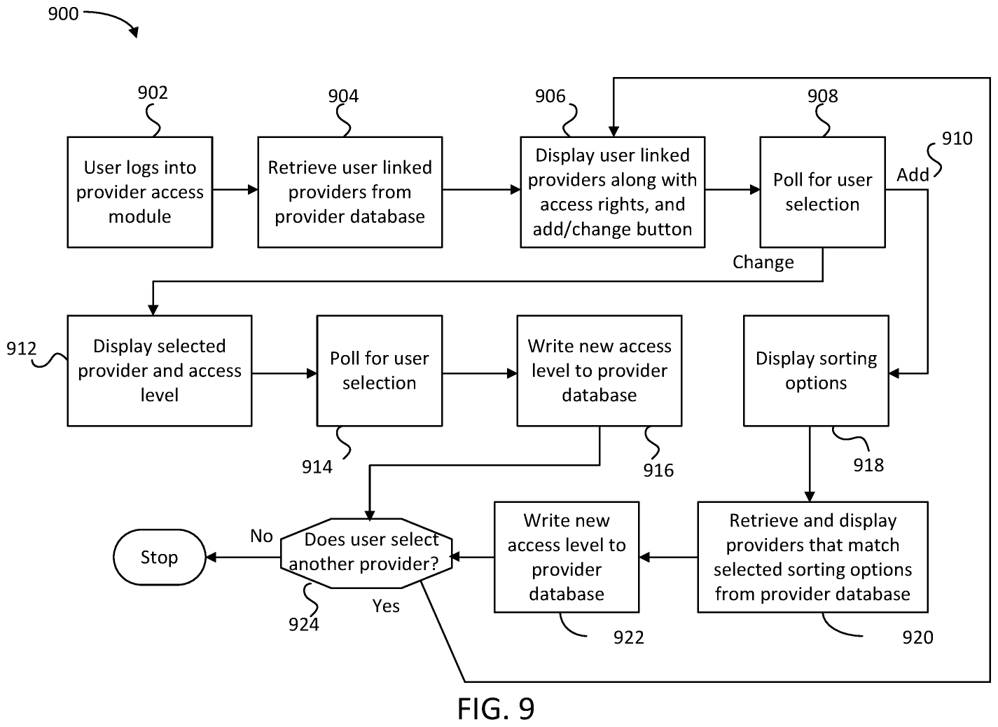

FIG. 9 illustrates a flowchart in a method 900 for operating a provider access module (e.g., provider access module 134), according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the user may login to the provider access module 134, at step 902. Upon logging-in, details about user linked providers may be retrieved from the provider database 124, at step 904. The user linked providers may indicate provider(s) having a data sharing relationship with the user. Details of the user linked providers along with their access rights may be then displayed on the user device 104, at step 906. In various embodiments, radio buttons for allowing to change access rights of currently linked providers and/or to add a new provider may also be displayed on the user device 104.

In accordance with various embodiments, user selection may be polled, at step 908. Further to the polling, if the user selects to change details/access rights of an existing provider, a current access level the provider has to the patient's data, type of data, and access in which circumstances is allowed, may be displayed, at step 912. Thereafter, the user selection may be polled for changing the access rights of the existing provider, at step 914. The user's adjustments may then be stored in the provider database 124, at step 916. In another case, while the user selects to add access rights at step 910, details of providers matching selected sorting options may be retrieved from the provider database 124 and may be displayed, at step 918. Step 920 may include retrieving and displaying providers that match selected sorting options from the provider database. Such details of providers may include location and provider type and may be displayed, at step 920. A new access right/level may be written (e.g., stored) for the provider, in the provider database 124, at step 922. If the user selects another provider at step 924, control may be transferred back to step 906, and the process may be terminated while the user does not select another provider.

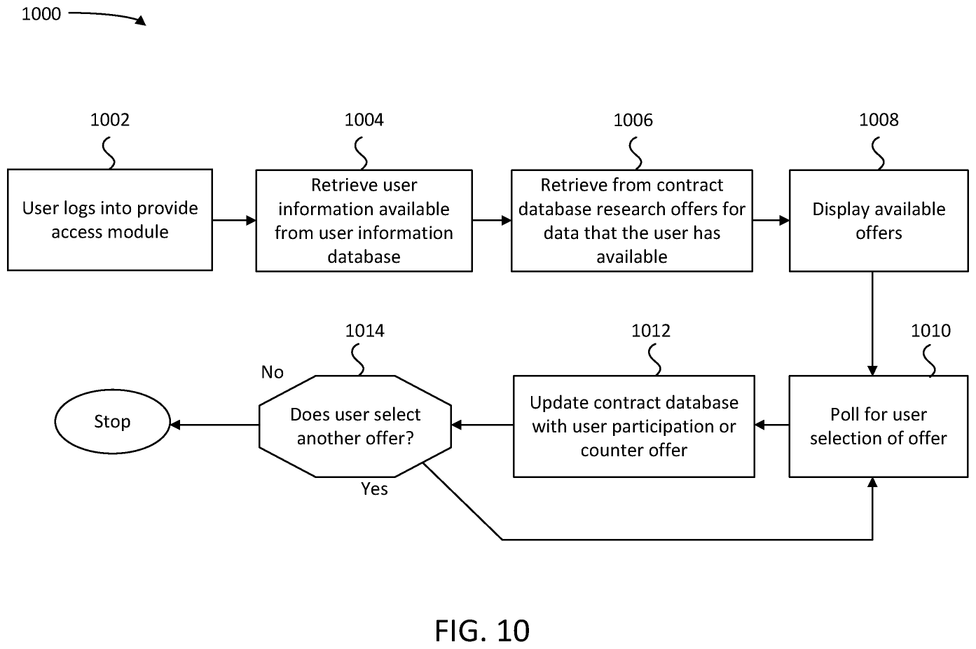

FIG. 10 illustrates a flowchart in a method 1000 for operating a research access module (e.g., research access module 136), according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the user may login to the research access module 136, at step 1002. Further to selection of research option, user information may be retrieved from the user information database 130, at step 1004. In various embodiments, type(s) of information the user may provide to a research organization, such as a glucometer or activity monitor, may also be retrieved. In various embodiments, the module may retrieve information from the provider database 124, at step 1006. The information may be related to research organizations that may be looking for data that the user can provide. Successively, available offers that the user can fulfill may be displayed, at step 1008. The user's selection may then be polled, at step 1010.

In accordance with various embodiments, the user may accept the current offer or may indicate a price at which he would provide the requested data, at step 1012. Such information may be updated in the contract database 128, at step 1012. Post completion of the first offer, the user may select another offer, at step 1014. Control may be transferred back to step 1010 while the user selects another process, or the process may be terminated while the user does not select another offer.

### Computer System

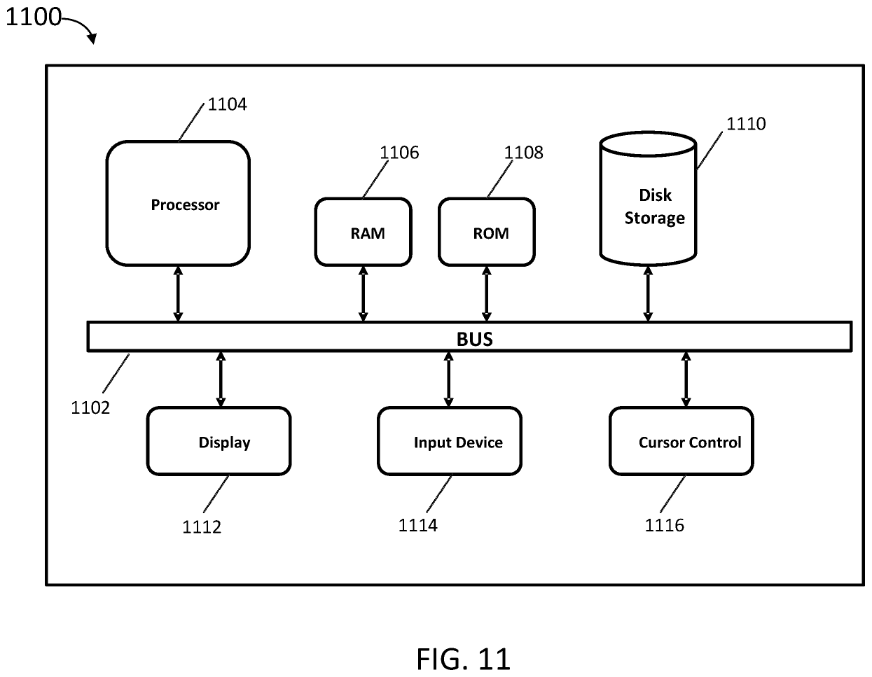

FIG. 11 is a block diagram that illustrates a computer system 1100, upon which embodiments, or portions of the embodiments, of the present teachings may be implemented. In various embodiments of the present teachings, computer system 1100 can include a bus 1102 or other communication mechanism for communicating information, and a processor 1104 coupled with bus 1102 for processing information. In various embodiments, computer system 1100 can also include a memory 1106, which can be a random access memory (RAM) or other dynamic storage device, coupled to bus 1102 for determining instructions to be executed by processor 1104. Memory 1106 also can be used for storing temporary variables or other intermediate information during execution of instructions to be executed by processor 1104. In various embodiments, computer system 1100 can further include a read-only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communicating information and command selections to processor 1104. Another type of user input device is a cursor control 1116, such as a mouse, a trackball, or cursor direction keys for communicating direction information and command selections to processor 1104 and for controlling cursor movement on display 1112. This input device 1114 typically has two degrees of freedom in two axes, a first axis (e.g., x) and a second axis (e.g., y), that allows the device to specify positions in a plane. However, it should be understood that input devices 1114 allowing for 3-dimensional (x, y, and z) cursor movement are also contemplated herein.

Consistent with certain implementations of the present teachings, results can be provided by computer system 1100 in response to processor 1104 executing one or more sequences of one or more instructions contained in memory 1106. Such instructions can be read into memory 1106 from another computer-readable medium or computer-readable storage medium, such as storage device 1110. Execution of the sequences of instructions contained in memory 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, and magnetic disks, such as storage device 1110. Examples of volatile media can include, but are not limited to, dynamic memory, such as memory 1106. Examples of transmission media can include, but are not limited to, coaxial cables, copper wire, and fiber optics, including the wires that include bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to a computer-readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, and the like.

It should be appreciated that the methodologies described herein including flow charts, diagrams, and the accompanying disclosure can be implemented using computer system 1100 as a standalone device or on a distributed network of shared computer processing resources such as a cloud computing network.

In accordance with various embodiments, the systems and methods described herein can be implemented using computer system 1100 as a standalone device or on a distributed network of shared computer processing resources such as a cloud computing network. As such, a non-transitory computer-readable medium can be provided in which a program is stored for causing a computer to perform the disclosed methods for identifying mutually incompatible gene pairs.

It should also be understood that the preceding embodiments can be provided, in whole or in part, as a system of components integrated to perform the methods described. For example, in accordance with various embodiments, the methods described herein can be provided as a system of components or stations for analytically determining novelty responses.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, their specific configuration, communication, and physical orientation with respect to each other. One skilled in the art should readily appreciate that these components can have various configurations and physical orientations (e.g., wholly separate components, units, and subunits of groups of components, different communication regimes between components).

Embodiments as disclosed herein include:

A. A computer-implemented method for managing access of a user's health information stored in a healthcare network includes configuring a health information exchange server implemented over a blockchain network to store a users' health information. The computer-implemented method also includes configuring the health information exchange server to communicate with a user device present in the healthcare network, providing, through a smart contract set by a user, an access of the user's health information to a provider, based on an access right assigned to the provider in the smart contract, and receiving a monetary value set for the access right.

B. A system for managing access of a user's health information stored in a healthcare network includes a memory circuit storing instructions, and one or more processors configured to execute the instructions. The instructions cause the system to configure a health information exchange server implemented over a blockchain network to store a users' health information, to configure the health information exchange server to communicate with a user device present in the healthcare network, to provide, through a smart contract set by a user, an access of the user's health information to a provider, based on an access right assigned to the provider in the smart contract, and to receive a monetary value set for the access right.

C. A computer-implemented method for accessing a patient's health information in a healthcare network, includes receiving, in a record access module, a request to access a data from a provider and verifying that the provider has authorization to access the data, based on a smart contract in a contracts database. The computer-implemented method also includes retrieving a contract factor from the smart contract, the contract factor based on the request to access the data, calculating a contract rate based on the contract factor, providing the contract rate to the provider for approval, providing the contract rate to the record access module when the provider approves the contract rate, and allowing the record access module to provide the provider an access to the data.

Each of embodiments A, B, and C may have one or more of the following additional elements in any combination: Element 1, wherein the provider includes at least one of an individual associated with a hospital, an insurance company, a Contract Research Organization, or a pharmaceutical company, further including verifying whether the hospital, the insurance company, the Contract Research Organization, or the pharmaceutical company is listed in a provider database, wherein the provider database is part of a blockchain database. Element 2, further including adjusting the smart contract with a modified rate based on the monetary value set for the access right. Element 3, further including adding the access of the user's health information in a blockchain string stored in a blockchain database. Element 4, wherein providing an access of the user's health information to a provider includes verifying that the provider has a blockchain string in a blockchain database associated with the healthcare network. Element 5, wherein providing an access of the user's health information includes requesting an authorization from the user. Element 6, further including adjusting at least one rule in the smart contract when the provider rejects to pay the monetary value. Element 7, further including deleting the smart contract when the user declines a permission to access the user's health information. Element 8, wherein receiving a monetary value for the access right includes receiving a cryptocurrency from the provider. Element 9, further including releasing a private key for the provider to decrypt the user's health information from a blockchain string stored in a blockchain database.

Each of embodiments A, B, and C may also have one or more of the following additional elements in any combination: Element 10, wherein the provider comprises at least one of an individual associated with a hospital, an insurance company, a Contract Research Organization, or a pharmaceutical company, and the one or more processors further execute instructions to verify whether the hospital, the insurance company, the Contract Research Organization, or the pharmaceutical company is listed in a provider database, wherein the provider database is part of a blockchain database. Element 11, wherein the one or more processors further execute instructions to adjust the smart contract with a modified rate based on the monetary value set for the access right. Element 12, wherein the one or more processors further execute instructions to include the access of the user's health information in a blockchain string stored in a blockchain database. Element 13, wherein to provide an access of the user's health information to a provider the one or more processors execute instructions to verify that the provider has a blockchain string in a blockchain database associated work.

Each of embodiments A, B, and C may also have one or more of the following additional elements in any combination: Element 14, further comprising presenting an offset from the contract database to the provider when the provider rejects the contract rate. Element 15, wherein verifying that the provider has authorization to access the data comprises verifying that the provider has a blockchain string in a blockchain database associated with the healthcare network. Element 16, further comprising adjusting the contract factor in the smart contract when the provider rejects the contract rate. Element 17, further comprising adding an access to the data by the provider to a blockchain string in a blockchain network.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for managing access of a user's health information stored in a healthcare network, the method comprising:
configuring a health information exchange server implemented over a blockchain network to store a users' health information;
configuring the health information exchange server to communicate with a user device present in the healthcare network;
providing, through a smart contract set by a user, an access of the user's health information to a provider, based on an access right assigned to the provider in the smart contract; and
receiving a monetary value set for the access right.

2. The computer-implemented method of claim 1, wherein the provider comprises at least one of an individual associated with a hospital, an insurance company, a Contract Research Organization, or a pharmaceutical company, further comprising verifying whether the hospital, the insurance company, the Contract Research Organization, or the pharmaceutical company is listed in a provider database, wherein the provider database is part of a blockchain database.

3. The computer-implemented method of any one of claims 1 and 2, further comprising adjusting the smart contract with a modified rate based on the monetary value set for the access right.

4. The computer-implemented method of any one of claims 1 through 3, further comprising including the access of the user's health information in a blockchain string stored in a blockchain database.

5. The computer-implemented method of any one of claims 1 through 4, wherein providing an access of the user's health information to a provider comprises verifying that the provider has a blockchain string in a blockchain database associated with the healthcare network.

6. The computer-implemented method of any one of claims 1 through 5, wherein providing an access of the user's health information comprises requesting an authorization from the user.

7. The computer-implemented method of any one of claims 1 through 6, further comprising adjusting at least one rule in the smart contract when the provider rejects to pay the monetary value.

8. The computer-implemented method of any one of claims 1 through 7, further comprising deleting the smart contract when the user declines a permission to access the user's health information.

9. The computer-implemented method of any one of claims 1 through 8, wherein receiving a monetary value for the access right comprises receiving a cryptocurrency from the provider.

10. The computer-implemented method of any one of claims 1 through 9, further comprising releasing a private key for the provider to decrypt the user's health information from a blockchain string stored in a blockchain database.

11. A system for managing access of a user's health information stored in a healthcare network, comprising:
a memory circuit storing instructions; and
one or more processors configured to execute the instructions to cause the system to:
configure a health information exchange server implemented over a blockchain network to store a users' health information;
configure the health information exchange server to communicate with a user device present in the healthcare network;
provide, through a smart contract set by a user, an access of the user's health information to a provider, based on an access right assigned to the provider in the smart contract; and
receive a monetary value set for the access right.

12. The system of claim 11, wherein the provider comprises at least one of an individual associated with a hospital, an insurance company, a Contract Research Organization, or a pharmaceutical company, and the one or more processors further execute instructions to verify whether the hospital, the insurance company, the Contract Research Organization, or the pharmaceutical company is listed in a provider database, wherein the provider database is part of a blockchain database.

13. The system of any one of claims 11 and 12, wherein the one or more processors further execute instructions to adjust the smart contract with a modified rate based on the monetary value set for the access right.

14. The system of any one of claims 11 through 13, wherein the one or more processors further execute instructions to include the access of the user's health information in a blockchain string stored in a blockchain database.

15. The system of any one of claims 11 through 14, wherein to provide an access of the user's health information to a provider the one or more processors execute instructions to verify that the provider has a blockchain string in a blockchain database associated work.

16. A computer-implemented method for accessing a patient's health information in a healthcare network, comprising:
receiving, in a record access module, a request to access a data from a provider;
verifying that the provider has authorization to access the data, based on a smart contract in a contracts database;
retrieving a contract factor from the smart contract, the contract factor based on the request to access the data;
calculating a contract rate based on the contract factor;
providing the contract rate to the provider for approval;
providing the contract rate to the record access module when the provider approves the contract rate; and
allow the record access module to provide the provider an access to the data.

17. The computer-implemented method of claim 16, further comprising presenting an offset from the contract database to the provider when the provider rejects the contract rate.

18. The computer-implemented method of any one of claims 16 and 17, wherein verifying that the provider has authorization to access the data comprises verifying that the provider has a blockchain string in a blockchain database associated with the healthcare network.

19. The computer-implemented method of any one of claims 16 through 18, further comprising adjusting the contract factor in the smart contract when the provider rejects the contract rate.

20. The computer-implemented method of any one of claims 16 through 19, further comprising adding an access to the data by the provider to a blockchain string in a blockchain network.
